(19) United States
(12) Patent Application Publication
(10) Pub. No.: US 2014/0234259 A1
(43) Pub. Date: Aug. 21, 2014

(54) Natural Intra-Vaginal Inserts to Control Imbalanced pH

(71) Applicant: Suzanah Juras, Moraga, CA (US)

(72) Inventor: Suzanah Juras, Moraga, CA (US)

(21) Appl. No.: 13/711,416

(22) Filed: Dec. 11, 2012

(51) Int. Cl. A61K 35/74

(52) U.S. Cl. CPC A61K 35/744
USPC 424/93.3

(57) ABSTRACT

A vaginal insert controls pH within a human vagina by use of various probiotics which include lactobacillus reuteri, lactobacillus rhamnosus, lactobacillus casei, lactobacillus plantarum, lactobacillus acidophilus, lactobacillus brevis, bifidobacterium lactis, bifidobacterium longum, lactobacillus paracasei, lactobacillus salivarius, and lactobacillus bulgaricus. In other embodiments, the use of aloe vera and aloe vera based derivatives are disclosed. Such derivatives include stabilized high molecular weight aloe vera gel polysaccharides (AVP), purified polysaccharide fractions, aloe succotrina, and aloe vera polysaccharide of Dalton weights in the range of 100,000 to 7 million. The disclosed embodiments may be applied or inserted in the form of gels, tablets, powders and other modalities.

### CROSS-REFERENCE TO RELATED APPLICATIONS

Not Applicable

### BACKGROUND OF THE INVENTION

### Field of the Invention

The present invention generally relates to a non-pharmaceutical intra-vaginal means and methods of correcting an imbalanced pH for a healthier vaginal ecosystem for women.

### BRIEF SUMMARY OF THE INVENTION

The present invention is a natural blend of approximately 11 probiotics intended for intra-vaginal use. An object of the present invention is to correct an imbalanced pH and to repopulate the delicate micro-flora of the vagina with strains of beneficial bacteria to create a healthier vaginal ecosystem. Embodiments include the use of aloe vera as well as the use of polysaccharide fractions prepared from aloe vera. Embodiments also use aloe succotriana or Fynbos aloe.

An object of the present invention is to use combinations of probiotics to generate hydrogen peroxide to obtain an optimal vaginal pH level. The disclosed combinations of probiotics have achieved unexpected results in restoring vaginal health. The use of lactobacilli creates an intra-vaginal chemical reaction producing hydrogen peroxide at the expense of unwanted bacteria and fungi.

The inhibition of growth of an unwanted bacterial species by the hydrogen peroxide generated by another bacterial species is a well-recognized mechanism of bacterial antagonism. Lactobacilli utilize flavoproteins, (a class of proteins that contain flavins and are involved in oxidation reactions in cells) which generally convert oxygen to hydrogen peroxide. This oxygen to hydrogen peroxide transformation mechanism generally results in the formation of hydrogen peroxide in amounts that are in excess of the capacity of the offending organism to degrade it. The hydrogen peroxide formed may inhibit or kill other members of the micro biota, particularly those that lack, or have low levels of hydrogen peroxide scavenging enzymes. Lactobacilli and other lactic-acid producing bacteria are among the organisms which can generate the hydrogen peroxide required for microbial antagonism.

Lactobacilli species are usually predominant in the vaginas of normal, healthy women who are without any active bacterial or fungal infections. Lactobacilli species are believed to regulate the growth of other vaginal flora. Researchers originally postulated that Haemophilus vaginalis, now known as Gardnerella vaginalis, was the sole cause of bacterial vaginosis (BV), because G. vaginalis was recovered from the vaginas of 92% of women with bacterial vaginosis and from no women without BV. However, the role of G. vaginalis in BV became less clear as researchers began to report a 30% to 40% prevalence of G. vaginalis among normal women.

More recently it has been reported that the prevalence and concentration of certain vaginal anaerobic bacteria, as well as that of G. vaginalis, are higher among those women with bacterial vaginosis than those without it. It was reported that the Lactobacilli were notably absent on vaginal smears for women with BV, and other researchers isolated Lactobacillus species less often from women with BV than from those women without BV.

Therefore, Lactobacilli play in important role in the homeostasis of the normal vaginal flora by producing hydrogen peroxide. An absence of hydrogen peroxide producing Lactobacillus species thus could allow an overgrowth of catalase-negative organisms, (an enzyme found in the blood and in most living cells that splits (catalyzes) hydrogen peroxide to yield oxygen and water) such as those found in high concentrations among women with BV. Because hydrogen peroxide is a potent antimicrobial agent, the role of hydrogen peroxide producing Lactobacilli in the prevention of bacterial vaginosis through the inhibition of the intravaginal growth of offending microorganisms is important in maintaining a healthy vaginal ecosystem.

In the present invention, the beneficial bacteria assist the vagina in restoring pH, which for a normal, healthy vagina is an acidic environment, typically between 3.8-4.5. pH balance is a fundamental component of vaginal health. As supported from the above, a properly balanced pH promotes a healthy vaginal ecosystem, and is key to help protect against pathogenic organisms like an overgrowth of bacteria or fungi. This in turn helps to correct the undesirable vaginal odor, itching, and discharge resulting from infections.

Active ingredients for the tablets, capsules and powder formulations include:

Lactobacillus reuteri—5 billion organisms per dose

Lactobacillus rhamnosus—17 billion organisms per dose

Lactobacillus casei—12 billion organisms per dose

Lactobacillus plantarum—8 billion organisms per dose

Lactobacillus acidophilus—5 billion organisms per dose

Lactobacillus brevis—3 billion organisms per dose

Bifidobacterium lactis—3 billion organisms per dose

Bifidobacterium longum—3 billion organisms per dose

Lactobacillus paracasei—500 million organisms per dose

Lactobacillus salivarius—500 million organisms per dose

Lactobacillus bulgaricus—250 million organisms per dose

In alternative embodiments, the active ingredients are measured by weight. The disclosed active ingredients are disclosed in approximate proportions and alternative proportions are contemplated.

Embodiments include the ingredients above and the use of aloe vera in the range of 40 to 60 milligrams per dose with a preferred embodiment of 57 milligrams per does. Embodiments include the ingredients above with the use of:
1. Stabilized aloe vera polysaccharides;
2. Stabilized high molecular weight aloe vera gel polysaccharides (AVP) as described in Human Colonic Bacteria Can Utilize Stabilized Aloe Vera Gel Polysaccharides and a Mixed Saccharide-Based Glyconutritinal Dietary Supplement, Advanced Ambrotose by Sinnot et al, a copy of which is attached to the IDS filed with this application.
3. Purified polysaccharide fractions designated as PAC-1, PAC-II and PAC-III prepared from Aloe vera L. var. Chinensis (Haw.) Berg. By membrane fractionation and gel filtration HPLC. The polysaccharide fractions having molecular weights of 10,00 kDA, 13,000 kDA, and 470 kDA, respectively as further described in Chemical and biological characterization of a polysaccharide biological response modifier from Aloe vera L. var. chinensis (Haw.) Berg published Jan. 22, 2004 in Glycobiology vol 14 no. 6 pp. 501-510, 204, a copy of which is attached to the IDS filed with this application.
4. Aloe vera polysaccharide of Dalton weights in the range of 100,000 to 7 million;
5. Aloeride polysaccharides of Dalton weights in the range of 100,000 to 7 million; and
6. Aloe succotrina.

The disclosed compounds may be applied in various forms and by various delivery methods. The invention contemplates one or more dissolving containers used hold the disclosed active ingredients. Such a system of delivery would allow for relatively smaller containers to be inserted into a vagina and for certain combinations of ingredients to mix intra vaginally. The described delivery method or product may sometimes be called “vaginal vitamins.”

The disclosed methods, products and systems present an advantage over the related art as only natural ingredients are used and such ingredients are used in an unobvious and artful combination.

These and other objects and advantages will be made apparent when considering the following detailed specification when taken in conjunction with the drawings.

### BRIEF DESCRIPTION OF THE DRAWINGS

FIG. 1 is chart of the main active ingredients for one embodiment.

### DETAILED DESCRIPTION OF EMBODIMENTS OF THE INVENTION

The following detailed description is directed to certain specific embodiments of the invention. However, the invention can be embodied in a multitude of different ways as defined and covered by the claims and their equivalents. In this description, reference is made to the drawings wherein like parts are designated with like numerals throughout.

Unless otherwise noted in this specification or in the claims, all of the terms used in the specification and the claims will have the meanings normally ascribed to these terms by workers in the art.

Unless the context clearly requires otherwise, throughout the description and the claims, the words “comprise,” “comprising” and the like are to be construed in an inclusive sense as opposed to an exclusive or exhaustive sense; that is to say, in a sense of “including, but not limited to.” Words using the singular or plural number also include the plural or singular number, respectively. Additionally, the words “herein,” “above,” “below,” and words of similar import, when used in this application, shall refer to this application as a whole and not to any particular portions of this application.

The above detailed description of embodiments of the invention is not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. For example, while steps are presented in a given order, alternative embodiments may perform routines having steps in a different order. The teachings of the invention provided herein can be applied to other systems, not only the systems described herein. The various embodiments described herein can be combined to provide further embodiments. These and other changes can be made to the invention in light of the detailed description.

Any and all the above references and U.S. patents and applications are incorporated herein by reference. Aspects of the invention can be modified, if necessary, to employ the systems, functions and concepts of the various patents and applications described above to provide yet further embodiments of the invention.

These and other changes can be made to the invention in light of the above detailed description. In general, the terms used in the following claims, should not be construed to limit the invention to the specific embodiments disclosed in the specification, unless the above detailed description explicitly defines such terms. Accordingly, the actual scope of the invention encompasses the disclosed embodiments and all equivalent ways of practicing

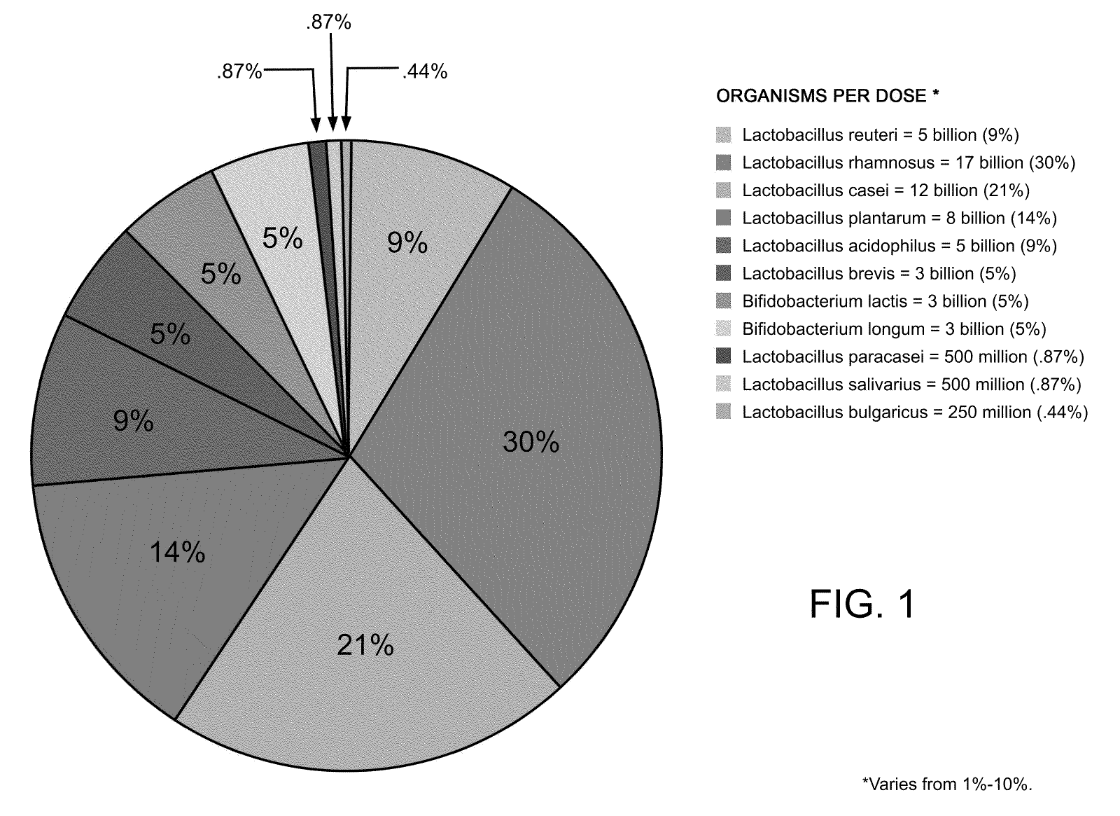

FIG. 1 depicts a chart of the main active ingredients for one embodiment. The chart shows one embodiment with amounts and percentages of organisms per dose. Amounts may vary between 1-10 percent. Disclosed ingredients include, but are not limited to the following ingredients in the following amounts per dosage. The total dosage amounts will vary.

Lactobacillus reuteri—4 to 7 billion organisms per dose

Lactobacillus rhamnosus—14 to 20 billion organisms per dose

Lactobacillus casei—8 to 14 billion organisms per dose

Lactobacillus plantarum—6 to 10 billion organisms per dose

Lactobacillus acidophilus—3 to 7 billion organisms per dose

Lactobacillus brevis—1 to 5 billion organisms per dose

Bifidobacterium lactis—1 to 5 billion organisms per dose

Bifidobacterium longum—1 to 5 billion organisms per dose

Lactobacillus paracasei—300 to 700 million organisms per dose

Lactobacillus salivarius—300 to 700 million organisms per dose

Lactobacillus bulgaricus—100 to 400 million organisms per dose

The use of other ingredients is contemplated as described above and includes the addition of 40 to 60 milligrams any of the following compounds:

1. Stabilized aloe vera polysaccharides;

2. Stabilized high molecular weight aloe vera gel polysaccharides (AVP).

3. Purified polysaccharide fractions designated as PAC-1, PAC-II and PAC-III prepared from Aloe vera L. var. Chinensis (Haw.) Berg.

4. Aloe vera polysaccharide of Dalton weights in the range of 100,000 to 7 million;

5. Aloeride polysaccharides of Dalton weights in the range of 100,000 to 7 million;

6. Aloe succotrina.

The use of aloe vera and the above referenced aloe vera derivatives

### Items

Disclosed embodiments include the following items:

Item 1. A vaginal insert to control imbalanced pH within a human vagina, the insert comprising:
a) Lactobacillus reuteri—4 to 7 billion organisms per dose;
b) Lactobacillus rhamnosus—14 to 20 billion organisms per dose;
c) Lactobacillus casei—8 to 14 billion organisms per dose;
d) Lactobacillus plantarum—6 to 10 billion organisms per dose;
e) Lactobacillus acidophilus—3 to 7 billion organisms per dose;
f) Lactobacillus brevis—1 to 5 billion organisms per dose;
g) Bifidobacterium lactis—1 to 5 billion organisms per dose;
h) Bifidobacterium longum—1 to 5 billion organisms per dose;
i) Lactobacillus paracasei—300 to 700 million organisms per dose;
j) Lactobacillus salivarius—300 to 700 million organisms per dose; and
k) Lactobacillus bulgaricus—100 to 400 million organisms per dose.

Item 2. The insert of item 1 further comprising aloe vera polysaccharide of Dalton weights in the range of 100,000 to 7 million and in the amount of 40 to 60 milligrams per dose.

Item 3. The insert of item 1 further comprising aloe vera polysaccharides in the amount of 40 to 60 milligrams per dose.

Item 4. The insert of item 1 further comprising stabilized aloe vera polysaccharides in the amount of 40 to 60 milligrams per dose.

Item 5. The insert of item 1 further comprising aloe vera in the amount of 40 to 60 milligrams per dose.

Item 6. The insert of item 1 further comprising aloeride polysaccharides of Dalton weights in the range of 100,000 to 7 million and in the amount of 40 to 60 milligrams per dose.

Item 7. The insert of item 1 further comprising aloe succotrina in the amount of 40 to 60 milligrams per dose.

Item 8. The insert of item 1 with the following ingredients in the following amounts:
a) Lactobacillus reuteri—4.5 to 5.5 billion organisms per dose;
b) Lactobacillus rhamnosus—16.5 to 17.5 billion organisms per dose;
c) Lactobacillus casei—11.5 to 12.5 billion organisms per dose;
d) Lactobacillus plantarum—7.5 to 8.5 billion organisms per dose;
e) Lactobacillus acidophilus—4.5 to 5.5 billion organisms per dose;
f) Lactobacillus brevis—2.5 to 3.5 billion organisms per dose;
g) Bifidobacterium lactis—2.5 to 3.5 billion organisms per dose;
h) Bifidobacterium longum—2.5 to 3.5 billion organisms per dose;
i) Lactobacillus paracasei—450 to 550 million organisms per dose;
j) Lactobacillus salivarius—450 to 550 million organisms per dose; and
k) Lactobacillus bulgaricus—200 to 300 million organisms per dose.

Item 9. A kit for making vaginal inserts to control pH, the kit comprising:
a) Lactobacillus reuteri—4 to 7 billion organisms per dose;
b) Lactobacillus rhamnosus—14 to 20 billion organisms per dose;
c) Lactobacillus casei—8 to 14 billion organisms per dose;
d) Lactobacillus plantarum—6 to 10 billion organisms per dose;
e) Lactobacillus acidophilus—3 to 7 billion organisms per dose;
f) Lactobacillus brevis—1 to 5 billion organisms per dose;
g) Bifidobacterium lactis—1 to 5 billion organisms per dose;
h) Bifidobacterium longum—1 to 5 billion organisms per dose;
i) Lactobacillus paracasei—300 to 700 million organisms per dose;
j) Lactobacillus salivarius—300 to 700 million organisms per dose; and
k) Lactobacillus bulgaricus—100 to 400 million organisms per dose.

Item 10. The kit of item 9 further comprising aloe vera polysaccharide of Dalton weights in the range of 100,000 to 7 million and in the amount of 40 to 60 milligrams per dose.

Item 11. The kit of item 9 further comprising aloe vera polysaccharides in the amount of 40 to 60 milligrams per dose.

Item 12. The kit of item 9 further comprising stabilized aloe vera polysaccharides in the amount of 40 to 60 milligrams per dose.

Item 13. The kit of item 9 further comprising aloe vera in the amount of 40 to 60 milligrams per dose.

Item 14. The kit of item 9 further comprising aloe succotrina in the amount of 40 to 60 milligrams per dose.

Item 15. A method of adjusting the pH within a human vagina, the method using:
a) Lactobacillus reuteri—4 to 7 billion organisms per dose;
b) Lactobacillus rhamnosus—14 to 20 billion organisms per dose;
c) Lactobacillus casei—8 to 14 billion organisms per dose;
d) Lactobacillus plantarum—6 to 10 billion organisms per dose;
e) Lactobacillus acidophilus—3 to 7 billion organisms per dose;
f) Lactobacillus brevis—1 to 5 billion organisms per dose;
g) Bifidobacterium lactis—1 to 5 billion organisms per dose;
h) Bifidobacterium longum—1 to 5 billion organisms per dose;
i) Lactobacillus paracasei—300 to 700 million organisms per dose;
j) Lactobacillus salivarius—300 to 700 million organisms per dose; and
k) Lactobacillus bulgaricus—100 to 400 million organisms per dose.

Item 16. The method of item 15 using the following ingredients and amounts:
a) Lactobacillus reuteri—4.5 to 5.5 billion organisms per dose;
b) Lactobacillus rhamnosus—16.5 to 17.5 billion organisms per dose;
c) Lactobacillus casei—11.5 to 12.5 billion organisms per dose;
d) Lactobacillus plantarum—7.5 to 8.5 billion organisms per dose;
e) Lactobacillus acidophilus—4.5 to 5.5 billion organisms per dose;
f) Lactobacillus brevis—2.5 to 3.5 billion organisms per dose;
g) Bifidobacterium lactis—2.5 to 3.5 billion organisms per dose;
h) Bifidobacterium longum—2.5 to 3.5 billion organisms per dose;
i) Lactobacillus paracasei—450 to 550 million organisms per dose;
j) Lactobacillus salivarius—450 to 550 million organisms per dose; and
k) Lactobacillus bulgaricus—200 to 300 million organisms per dose.

Item 17. The method of item 15 also using aloeride polysaccharides of Dalton weights in the range of 100,000 to 7 million and in the amount of 40 to 60 milligrams per dose.

Item 18. The method of item 15 also using aloe vera in the amount of 40 to 60 milligrams per dose.

Item 19. The method of item 15 also using purified polysaccharide fractions derived from aloe vera in the amount of 40 to 60 milligrams per dose.

Item 20. The method of item 15 also using aloe succotrina in the amount of 40 to 60 milligrams per dose.

What is claimed is:

1. An vaginal insert to control imbalanced pH, the insert comprising:
lactobacillus reuteri, 4 to 7 billion organisms per dose;
lactobacillus rhamnosus, 14 to 20 billion organisms per dose;
lactobacillus casei, 8 to 14 billion organisms per dose;
lactobacillus plantarum, 6 to 10 billion organisms per dose;
lactobacillus acidophilus, 3 to 7 billion organisms per dose;
lactobacillus brevis, 1 to 5 billion organisms per dose;
bifidobacterium lactis, 1 to 5 billion organisms per dose;
bifidobacterium longum, 1 to 5 billion organisms per dose;
lactobacillus paracasei, 300 to 700 million organisms per dose;
lactobacillus salivarius, 300 to 700 million organisms per dose; and
lactobacillus bulgaricus, 100 to 400 million organisms per dose.

2. The insert of claim 1 further comprising aloe vera polysaccharide of Dalton weights in the range of 100,000 to 7 million and in the amount of 40 to 60 milligrams per dose.

3. The insert of claim 1 further comprising aloe vera polysaccharides in the amount of 40 to 60 milligrams per dose.

4. The insert of claim 1 further comprising stabilized aloe vera polysaccharides in the amount of 40 to 60 milligrams per dose.

5. The insert of claim 1 further comprising aloe vera in the amount of 40 to 60 milligrams per dose.

6. The insert of claim 1 further comprising aloeride polysaccharides of Dalton weights in the range of 100,000 to 7 million and in the amount of 40 to 60 milligrams per dose.

7. The insert of claim 1 further comprising aloe succotrina in the amount of 40 to 60 milligrams per dose.

8. The insert of claim 1 with the following ingredients in the following amounts:
lactobacillus reuteri, 4.5 to 5.5 billion organisms per dose;
lactobacillus rhamnosus, 16.5 to 17.5 billion organisms per dose;
lactobacillus casei, 11.5 to 12.5 billion organisms per dose;
lactobacillus plantarum, 7.5 to 8.5 billion organisms per dose;
lactobacillus acidophilus, 4.5 to 5.5 billion organisms per dose;
lactobacillus brevis, 2.5 to 3.5 billion organisms per dose;
bifidobacterium lactis, 2.5 to 3.5 billion organisms per dose;
bifidobacterium longum, 2.5 to 3.5 billion organisms per dose;
lactobacillus paracasei, 450 to 550 million organisms per dose;
lactobacillus salivarius, 450 to 550 million organisms per dose; and
lactobacillus bulgaricus, 200 to 300 million organisms per dose.

9. A kit for making vaginal inserts to control pH, the kit comprising:
lactobacillus reuteri, 4 to 7 billion organisms per dose;
lactobacillus rhamnosus, 14 to 20 billion organisms per dose;
lactobacillus casei, 8 to 14 billion organisms per dose;
lactobacillus plantarum, 6 to 10 billion organisms per dose;
lactobacillus acidophilus, 3 to 7 billion organisms per dose;
lactobacillus brevis, 1 to 5 billion organisms per dose;
bifidobacterium lactis, 1 to 5 billion organisms per dose;
bifidobacterium longum, 1 to 5 billion organisms per dose;
lactobacillus paracasei, 300 to 700 million organisms per dose;
lactobacillus salivarius, 300 to 700 million organisms per dose; and
lactobacillus bulgaricus, 100 to 400 million organisms per dose.

10. The kit of claim 9 further comprising aloe vera polysaccharide of Dalton weights in the range of 100,000 to 7 million and in the amount of 40 to 60 milligrams per dose.

11. The kit of claim 9 further comprising aloe vera polysaccharides in the amount of 40 to 60 milligrams per dose.

12. The kit of claim 9 further comprising stabilized aloe vera polysaccharides in the amount of 40 to 60 milligrams per dose.

13. The kit of claim 9 further comprising aloe vera in the amount of 40 to 60 milligrams per dose.

14. The kit of claim 9 further comprising aloe succotrina in the amount of 40 to 60 milligrams per dose.

15. A method of adjusting the pH within a human vagina, the method using:
lactobacillus reuteri—4 to 7 billion organisms per dose;
lactobacillus rhamnosus—14 to 20 billion organisms per dose;
lactobacillus casei—8 to 14 billion organisms per dose;
lactobacillus plantarum—6 to 10 billion organisms per dose;
lactobacillus acidophilus—3 to 7 billion organisms per dose;
lactobacillus brevis—1 to 5 billion organisms per dose;
bifidobacterium lactis—1 to 5 billion organisms per dose;
bifidobacterium longum—1 to 5 billion organisms per dose;
lactobacillus paracasei—300 to 700 million organisms per dose;
lactobacillus salivarius—300 to 700 million organisms per dose; and
lactobacillus bulgaricus—100 to 400 million organisms per dose.

16. The method of claim 15 using the following ingredients and amounts:
lactobacillus reuteri—4.5 to 5.5 billion organisms per dose;
lactobacillus rhamnosus—16.5 to 17.5 billion organisms per dose;
lactobacillus casei—11.5 to 12.5 billion organisms per dose;
lactobacillus plantarum—7.5 to 8.5 billion organisms per dose;
lactobacillus acidophilus—4.5 to 5.5 billion organisms per dose;
lactobacillus brevis—2.5 to 3.5 billion organisms per dose;
bifidobacterium lactis—2.5 to 3.5 billion organisms per dose;
bifidobacterium longum—2.5 to 3.5 billion organisms per dose;
lactobacillus paracasei—450 to 550 million organisms per dose;
lactobacillus salivarius—450 to 550 million organisms per dose; and
lactobacillus bulgaricus—200 to 300 million organisms per dose.

17. The method of claim 15 also using aloeride polysaccharides of Dalton weights in the range of 100,000 to 7 million and in the amount of 40 to 60 milligrams per dose.

18. The method of claim 15 also using aloe vera in the amount of 40 to 60 milligrams per dose.

19. The method of claim 15 also using purified polysaccharide fractions derived from aloe vera in the amount of 40 to 60 milligrams per dose.

20. The method of claim 15 using aloe succotrina in the amount of 40 to 60 milligrams per dose.
